(19) United States
(12) Patent Application Publication
(10) Pub. No.: US 2016/0007988 A1
(43) Pub. Date: Jan. 14, 2016

(54) ANCHOR INSERTION DEVICE WITH WINDOW SPIKE TIP

(71) Applicant: Smith & Nephew, Inc., Memphis, TN (US)

(72) Inventors: Wei Li Fan, Boston, MA (US); Timothy Young, Natick, MA (US)

(73) Assignee: Smith & Nephew, Inc., Memphis, TN (US)

(21) Appl. No.: 14/770,267

(22) PCT Filed: Feb. 25, 2014
(86) PCT No.: PCT/US2014/018342

§ 371 Date: Aug. 25, 2015

Related U.S. Application Data
(60) Provisional application No. 61/769,411, filed on Feb. 26, 2013.

(51) Int. Cl. A61B 17/04

(52) U.S. Cl. CPC A61B 17/0401; A61B 2017/0409; A61B 2017/00907

(57) ABSTRACT

A surgical device includes a generally elongated shaft having a cannulated interior dimensioned for slidably receiving an anchor. A distal end of the shaft includes a cutaway portion and a window. The window may be positioned within the cutaway portion or overlie at least a portion of the distal end of the shaft including the cutaway portion. The window may be transparent or partially-transparent, allowing the window to provide lateral support as well as visualization of an anchor sliding through and/or emerging from the distal end of the shaft. The cutaway portion may be open on at least one side (e.g., a distal-facing side).

### CROSS-REFERENCE TO RELATED APPLICATION(S)

This is a U.S. national phase application and claims priority to International Application No. PCT/US14/18342, filed on Feb. 25, 2014. The PCT/US14/18342 application claims the benefit of U.S. Provisional Application No. 61/769,411, filed on Feb. 26, 2013, and entitled, “Anchor Insertion Device With Window Spike Tip,” the entire teachings of the above applications are incorporate herein by reference.

### BACKGROUND

Medical devices and methods for attaching connective tissue to bone, such as soft tissue and prosthetic elements, are employed for surgical repair. Of particular interest, especially in sports medicine procedures, are suture anchors. A suture anchor is typically inserted into and fixed in a bore hole drilled into a bone at a surgical repair site. Sutures are typically coupled to the anchor and are used to secure the soft tissue to the bone in order to affect the repair. For many repair procedures, accurate placement of suture anchors in bone promotes positive surgical outcomes, and typically requires substantial skill on the part of the surgeon.

In practice, accurate placement of the bore holes and suture anchors can be particularly challenging when repair is performed arthroscopically, as both access to, and visibility of, an arthroscopic surgical site may be more limited than is the case with open surgical procedures. For example, accurately drilling bore holes and placing suture anchors into these holes, at certain joint areas of the body, can be difficult for even an experienced surgeon. Such difficulties may result from either inability of the delivery devices to reach a preferred anchor delivery point, or inability to achieve the preferred anchor trajectory, or both.

### SUMMARY

In an embodiment, a surgical device for bone anchor insertion employs a spike tip guide that has a window at the distal tip for visualization purposes. The window spike tip defines a delivery end of a surgical instrument for inserting a surgical anchor employing a cylindrical insertion member, typically a rotatable insertion shaft that maintains aggressive cutting teeth and closes an open portion of the shaft between the cutting teeth with transparent material to provide a continuous, flush surface around the cylinder. The proposed approach therefore provides a visualization means located at a “spike” tip defined by the teeth of the anchor insertion guide device where the visualization means may take the form of a transparent window element or a sectioned out cylinder closing the open side of the spike tip thus allowing the use of aggressive teeth and also providing lateral support for preventing the misalignment of a surgical anchor or “plug” during insertion.

In an embodiment, a surgical device includes a generally elongated shaft having a cannulated interior or bore. The shaft and cannula extend along a longitudinal axis between a proximal end and a distal end of the device. The cannulated interior of the shaft may be dimensioned for slidably receiving an anchor or instrumentation therein. The device further includes a proximal end responsive to surgical control for insertion of the insertion shaft into a surgical site along an axis defined by the cannulated interior. The device also includes a window positioned at the distal end of the shaft in visual communication with the cannulated interior of the shaft. The window provides lateral support to an anchor and/or instrumentation sliding through the distal end of the shaft.

In an embodiment, the surgical device may further include one or more of the following, alone or in any combination. A cutaway portion may be formed in the distal end of the shaft. The cutaway portion may open on at least one side. The at least one open side may include a distal-facing side. Alternatively, the window may be a cutaway portion, formed in the distal end of the shaft, bounded on all lateral sides by the shaft. The distal edge of the window may be spaced by a selected distance from the distal end of the shaft. The window may form at least a portion of an outer surface of the shaft. The window may be flush with at least a portion of the outer surface of the shaft. The window may be formed from a material that covers at least a portion of the cutaway portion. The window may be transparent or partially-transparent. The window may be positioned within the cutaway portion of the shaft and the thickness of the window may be equal to a wall thickness of the shaft. The window may be a tubular structure dimensioned to overlie at least a portion of the distal end of the shaft including the cutaway portion. The tubular window may be flush with at least a portion of an outer surface of the shaft proximal to the cutaway portion. The diameter of the distal portion of the shaft including the cutaway portion and the tubular window overlying the shaft may be approximately equal to the diameter of the shaft proximal to the cutaway portion. The shaft may further include a plurality of teeth formed in the distal end of the shaft. The teeth may be continuous with the cutaway region and form an edge defined by acutely angled edges of the cutaway region.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1 is a schematic illustration of an insertion device having a cutaway or void portion;

FIG. 2 is a schematic illustration of an embodiment of an improved insertion device having a window in an open-sided cutaway portion;

FIGS. 3A and 3B show an alternate embodiment of the insertion device having a closed-sided, oval cutaway portion;

FIGS. 4A and 4B show a further alternate embodiment of the insertion device having a closed-sided, rectangular cutaway portion; and

FIG. 5 shows an alternate embodiment of the insertion device employing a circumferential clear cover.

### DETAILED DESCRIPTION

Embodiments of the disclosure will now be discussed with regards to the Figures. It may be understood that the term “distal” will refer to a portion of an object located closest to a patient's anatomy while the term “proximal” will refer to a portion of an object located farther from the patient's anatomy than the distal portion thereof.

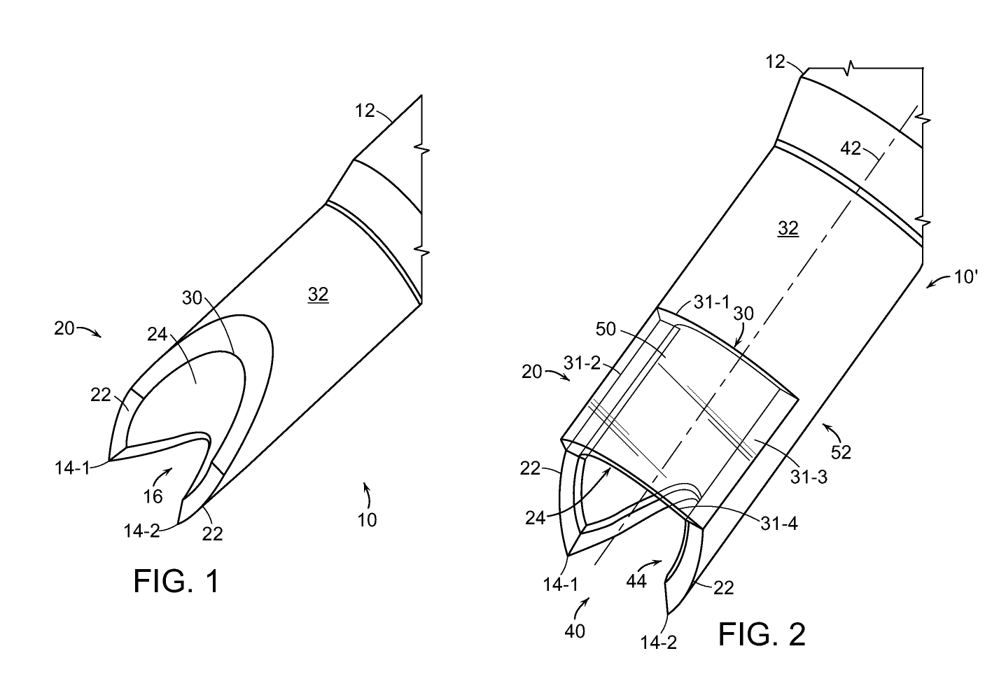

Referring to FIG. 1, an insertion device 10 for a bone anchor includes an insertion shaft 12 having one or more “spike” tips or teeth 14-1, 14-2 (14 generally) characterized by an acute angle 16 in a distal end 20 of the insertion shaft 12 forming sharp biting edges 22 adjacent to a void 24 formed by the sharp angle of the biting edges 22. Many surgeons prefer the benefits encapsulated in a spike tip guide, including highly aggressive teeth and visualization. Designs of the spike tip guide leave the toothless side of the tip very open in order to obtain the preferred benefits (see below). This cutaway portion or side 30 of a wall 32 of the shaft makes it easier for an anchor that is being inserted through the guide to “skive” out due to misalignment with an axis 42 of the shaft 12 and non-flush insertion from a cannulated interior 44 of the insertion shaft 12.

Referring to FIG. 2, an embodiment of an improved insertion device 10′ is illustrated. The insertion device 10′ includes a shaft or guide 12 which is generally elongate and possesses a cannulated interior or bore 40. The bore 40 is dimensioned to slidably receive a bone anchor and associated instrumentation, as discussed in greater detail below. The shaft 12 extends along the axis 42 (e.g., a longitudinal axis of the device 10′) between a proximal end (not shown) and the distal end 20. The proximal end is responsive to surgical control, allowing the insertion shaft 12 to be inserted into a surgical site along the longitudinal axis 42. The distal end 20 of the shaft 12 may include the one or more spike tips or teeth 14 (e.g., 14-1, 14-2) having biting edges 22.

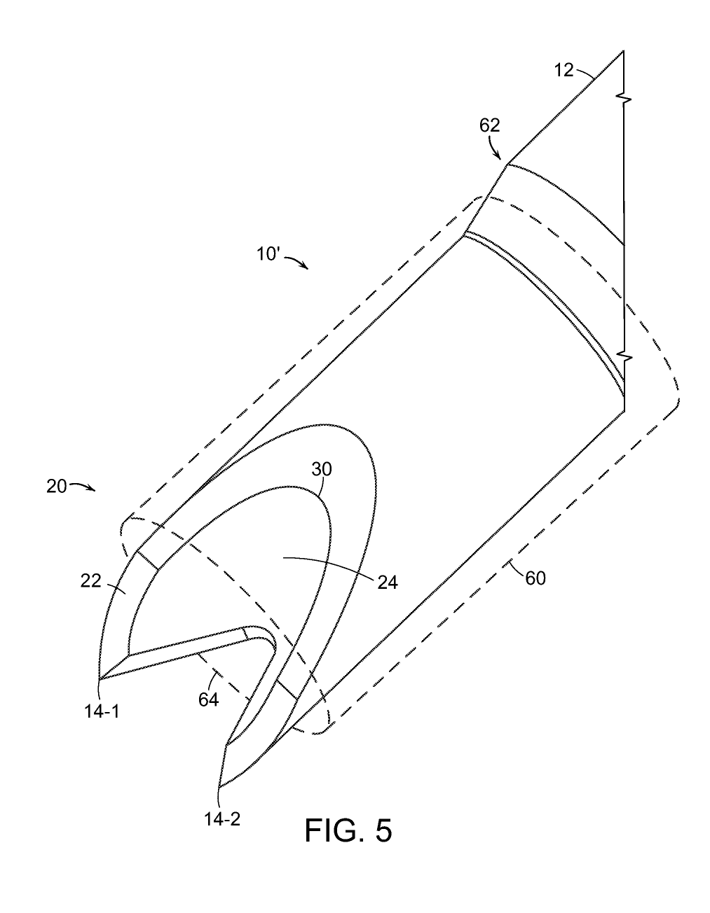

The device 10′ may further include a cutaway portion 30 and a window 50. The cutaway portion 30 formed through a wall 32 of the distal end 20 of the shaft 12, in communication with the bore 40. The cutaway portion 30 may further form the teeth 14 and the plurality of biting edges 22 of extending through the wall 32 at acute angles. The window 50 may be positioned at the distal end 20 of the shaft 12 and formed from a material that covers at least a portion of the cutaway portion 30, as discussed below. For example, in an embodiment, the window may be positioned within at least a portion of the cutaway portion, as illustrated in FIG. 2. In alternative embodiments, discussed in greater detail below with respect to FIG. 5, the window 50 may be formed as a tubular structure surrounding the distal end of the shaft, including the cutout region. So configured, the window 50 intersects the bore 40, allowing visualization of objects positioned within the bore 40 at the distal end 20 of the shaft 12 (e.g., bone anchors, and/or instrumentation attached thereto). For example, the window 50 may be employed to monitor the position and/or alignment of the anchor during passage through the distal end 20 of the device 10′ and alignment of the anchor upon emerging from the distal end 20 of the device 10′.

The window 50 further provides lateral support to an anchor positioned within the bore 40. For example, the window 50 may form at least a portion of the outer surface of the shaft (e.g., at least a portion of the outer surface of the shaft within the cutaway portion 30) and prevents biasing or force away from the axis 42 which can cause “skiving” or uneven insertion in conventional approaches.

In an embodiment, the window 50 may be formed from a transparent material. However, in alternative embodiments, the window may be formed from a partially or semi-transparent material. Examples of transparent and partially transparent materials may include, but are not limited to, plastics and glasses.

In certain embodiments, the window 50 may be dimensioned for positioning within at least a portion of the cutaway portion 30 of the shaft 12. For example, as illustrated in FIG. 2, the window 50 extends across the entire width of the cutaway portion 30 (e.g., between sides 31-2 to 31-3). In further embodiments, the window 50 may be dimensioned such that a distal end is spaced by a selected distance from the distal end of the shaft. In further embodiments, as illustrated in FIG. 2, this distance may be selected such that the distal edge of the window is positioned proximal to the biting edges 22 and teeth 14. So dimensioned, the window 50 does not engage surfaces (e.g., bone) along with the teeth 14 and/or biting edges 22 or experience the degree of wear or cutting forces exerted upon the teeth 14 and/or biting edges 22.

However, it may be understood that, in alternative embodiments, the window may adopt different geometries. For example, in one embodiment, the window may be dimensioned to extend distally with respect to the proximal end of the biting edges and/or the teeth. In such embodiments, the distal end of the window may be further formed to facilitate biting (e.g., formed with a sharp or pointed edge). In further embodiments, the window may be formed from a material having relatively high resistance to wear and/or strength suitable for use as a biting edge and/or tooth. In other embodiments, the window may cover only a portion of the width of the cutaway portion (e.g., from side 31-2 to a position short of side 31-3).

As discussed above, in certain embodiments, the window may be continuous and form a continuous outer surface with the shaft. However, in alternative embodiments, the window may be formed in two or more pieces. Such pieces may be dimensioned so as to form a continuous outer surface with the shaft. In alternative embodiments, such pieces may be discontinuous, dimensioned to cover a desired portion of the cutaway portion and having one or more spaces there-between.

In operation of the insertion device 10′, the elongated insertion shaft 12 receives an anchor and associated instrumentation (not shown) within the bore 40. Examples of such instrumentation may include, but is not limited to, obturators, drills, and anchor insertion devices. For example, in certain embodiments, the anchor may be mounted upon an elongate inserter and loaded within the bore 40 at the proximal end of the shaft 12. The inserter may also be dimensioned for receipt within the bore 40 and employed to urge the anchor from the proximal end to the distal end 20 of the shaft 12. The proximal end of the shaft 12 may be responsive to surgical control for insertion of the insertion shaft 12 into a surgical site along the axis 42 defined by the bore 40. Concurrently, the window 50 at the distal end 20 is adapted for visual communication with objects passing within the bore 40 at the distal end 20 of the shaft 12 (e.g., the received anchor and/or instrumentation) and provides lateral support thereto.

The window 50 may be attached to the shaft 12 by any suitable engagement mechanism. Examples of such engagement mechanisms may include, but are not limited to, one or more of adhesives, frictional engagements (e.g., overmolding), compressive engagements (i.e. snap-in) and mechanical interlocking (e.g., grooves, protrusions, and the like). Such engagement mechanisms may be present on one or more surfaces of the window 50 and the shaft 12 (e.g., lateral faces of the wall 32 and/or the window 50).

With continued reference to the embodiment of FIG. 2, a transparent or partially transparent window material is provided in the cutaway portion 30 and attached to the shaft 12. For example, as illustrated in FIG. 2, the cutaway portion 30 may be generally rectangular and bounded on three sides 31-1, 31-2, 31-3 by the wall 32 of the shaft 12. A fourth side of the cutaway portion 30 is open (e.g., distal-facing side 31-4). As a result, this fourth side defines an insertion end of the insertion device 10′ that is positioned adjacent to the cutting edge 22, allowing for visibility at the biting surface. Thus, the distal end 20 of the cutting tool 10′ has a rotary circumference partially defined by the cutting edge 22 and partially defined by the distal side of the window 50 (e.g., side 31-4).

While the embodiment of FIG. 2 illustrates a cutaway portion and window having four sides, it may be understood that, in alternative embodiments, the cutaway portion and/or the window may be formed in other shapes having greater or fewer sides. Furthermore, the window may be secured to the shaft at any one or more of the sides of the shaft wall abutting the window.

While the embodiment of FIG. 2 illustrates a cutaway portion having a distally-facing open side, it may be further understood that, in alternative embodiments, the cutaway portion may be open on a different side.

In certain embodiments, the window 50 may be dimensioned such that it is be flush with at least a portion of the outer surface 52 of the wall 32 defining the shaft 12. For example, the thickness of the window 50 may be approximately equal to the thickness of the wall 32 of the shaft 12. Thus, when the window 50 is positioned within the cutaway portion 30, the window 50 forms a continuous, flush surface with the outer surface 52 of the shaft 12 adjacent the cutaway portion 30. Such a flush geometry may inhibit the window 50 from undesirably engaging a patient's anatomy during insertion of the device 10′ (e.g., cutting or snagging) and allow the device 10′ to slide smoothly through a patient's anatomy.

In alternative embodiments, the thickness of the window may be less than or greater than the thickness of the wall of the shaft. When the thickness of the window is less than that of the wall of the shaft and the window is mounted within the cutaway portion, it may be recessed below the outer surface of the wall of the shaft. When the thickness of the window is greater than that of the wall of the shaft and the window is mounted within the cutaway portion, it may extend above the outer surface of the wall of the shaft.

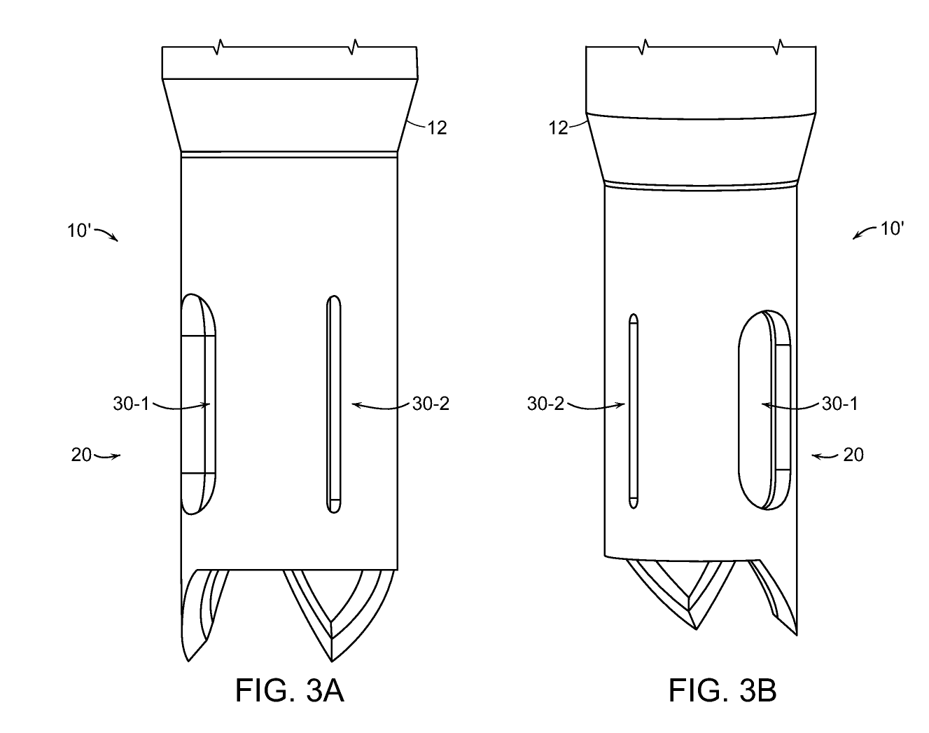

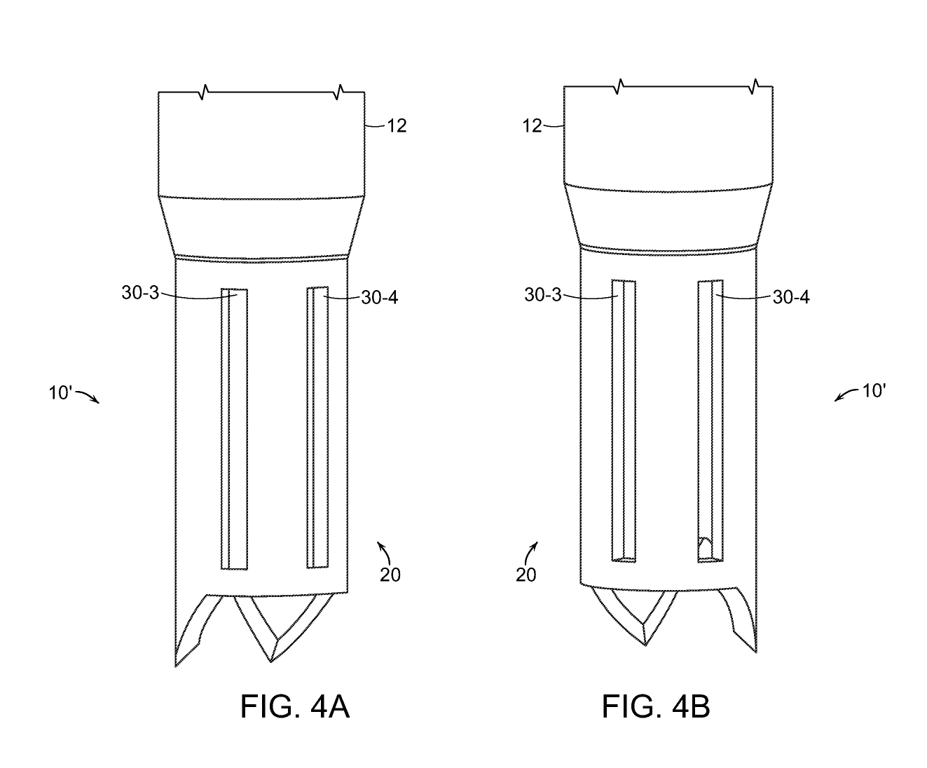

In alternative embodiments, with reference to FIGS. 3A-3B and 4A-4B, the device 10′ may be formed with a cutaway portion formed as a framed or closed-sided portal (e.g., 30-1, 30-2, 30-3, 30-4, collectively 30′) enclosed by the lateral sides of the wall 32 of the shaft 12 and in communication with the bore 40. In certain embodiments, the cutaway portion 30′ may provide sufficient visualization of the bore 40 and mechanical support for anchors, instrumentation, etc. passing there-through that it functions as both the cutaway and window, as discussed above with regards to FIG. 2. So configured, the rotary circumference of the distal end 20 is defined entirely by the biting edge 22 of the insertion device 10′. The cutaway portion 30′may be formed in any closed-sided shape, including, but not limited to, oval, rectangular, and elongated.

For example, FIGS. 3A and 3B show an embodiment of the insertion device 10′ having a plurality of cutaway portions 30-1, 30-2 formed in the shape of an oval cutout at the distal end 20. FIGS. 4A and 4B show a further alternate embodiment having a plurality of cutaway portions 30-3, 30-4 formed in the shape of a rectangular cutout at the distal end 20.

In other embodiments of the device 10′ having closed-sided cutaway portions (e.g., 30′), a window material may be positioned within at least a portion of one or more of the cutaway portions, as discussed above. Such window materials may be flush with the outer surface of the shaft, recessed below the outer surface of the shaft, or extend above the outer surface of the shaft.

Advantages of the approach of FIGS. 1, 2, 3A-B and 4A-B, as compared to existing insertion devices include allowing for greater visibility, preventing an anchor sliding within the bore 40 from skiving out, and permitting teeth (biting edges 22) to be used for facilitating engagement of the inserter 10′ with bone. FIG. 5 shows an alternate embodiment of the insertion device 10′, employing a circumferential window cover 60 that encircles or overlies at least a portion of the distal end 20 of the shaft 12 including the cutaway portion 30. The circumferential cover window 60 takes the shape of a tube-like segment adapted to slide over the distal end 20 of the shaft 12. The tubular structure of the window cover 60 provides a continuous covering surface around the distal end 20 and defines a transparent or partially-transparent region having a circumferential edge 64 at a distal end and the cutaway portion 30. The window cover 60 may be combined with any of the above-disclosed embodiments of the device 10′ having an open-sided cutaway portion (e.g., FIGS. 2, 5) or a closed-sided cutaway portion (e.g., FIGS. 3, 4).

As discussed above with respect to the window 50, the window cover 60 may be secured to the shaft 12 by adhesive, frictional, compressive, or other mechanical engagement. For example, in one embodiment, the window cover may be formed from an elastically deformable material (e.g., a polymer), allowing the window cover to expand and stretch slightly over the distal end 20. In an alternative embodiment, the window cover may be molded over the shaft. In further embodiments, the window cover may be secured to the distal end of the shaft by an adhesive or a crimp or ridge.

In certain embodiments, the window cover 60 may be flush with at least a portion of the outer surface of the shaft 12 (e.g., an outer surface of the shaft 12 proximal to the cutaway portion 30). For example, the shaft 12 may include a tapered portion 62 proximal to the cutaway portion 30. The tapered portion 62 reduces the diameter of the shaft 12 towards the distal end 20 including the cutaway portion 30 as compared to the diameter of the shaft 12 proximal to the tapered portion 62. In certain embodiments, the tapered portion 62 may reduce the diameter of the distal end of the shaft 12 that includes the cutaway portion 30 by an amount approximately equal to the thickness of the window cover 60. That is to say, the diameter of the distal portion 20 of the shaft 12 including the cutaway portion 30 and the window cover 60 overlying the shaft 12 may be approximately equal to the diameter of the shaft 12 proximal to the cutaway portion 60 and the tapered portion 62. In this manner, the tapered portion 62 compensates for the thickness of the cover 60 to maintain an approximately constant diameter along the length of the shaft 12. In alternative embodiments, the tapered portion may be omitted and the portion of the distal end of the shaft including the overlying window cover may possess a diameter greater than the portion of the shaft proximal to the cutaway portion.

The terms comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

What is claimed is:

1. A surgical device comprising:
a generally elongated shaft having a cannulated interior extending along a longitudinal axis between a proximal end and a distal end, the cannulated interior dimensioned for slidably receiving an anchor or instrumentation therein;
a proximal end responsive to surgical control for insertion of the insertion shaft into a surgical site along an axis defined by the cannulated interior; and
a window positioned at the distal end of the shaft in visual communication with the cannulated interior of the shaft, the window further providing lateral support to an anchor sliding through the distal end of the shaft.

2. The surgical device of claim 1, further comprising a cutaway portion formed in the distal end of the shaft, wherein the window is formed from a material that covers at least a portion of the cutaway portion.

3. The surgical device of claim 2, wherein the window is transparent.

4. The surgical device of claim 2, wherein the window is partially transparent.

5. The surgical device of claim 1, wherein the cutaway portion is open on at least one side.

6. The surgical device of claim 5, wherein the at least one open side of the cutaway portion includes a distal-facing side.

7. The surgical device of claim 1, wherein the window comprises a cutaway portion formed in the distal end of the shaft that is bounded on all lateral sides by the shaft.

8. The surgical device of claim 6, wherein a distal edge of the window is spaced by a selected distance from the distal end of the shaft.

9. The surgical device of claim 1, wherein the window forms a portion of an outer surface of the shaft.

10. The surgical device of claim 9, wherein the window is flush with at least a portion of the outer surface of the shaft.

11. The surgical device of claim 10, wherein the window is positioned within the cutaway portion of the shaft and wherein the thickness of the window is equal to a wall thickness of the shaft.

12. The surgical device of claim 1, wherein the window comprises a tube dimensioned to overlie at least a portion of the distal end of the shaft including the cutaway portion.

13. The surgical device of claim 12, wherein the window is flush with at least a portion of the outer surface of the shaft proximal to the cutaway portion.

14. The surgical device of claim 13, wherein the diameter of the distal portion of the shaft including the cutaway portion and the tubular window overlying the shaft is approximately equal to the diameter of the shaft proximal to the cutaway portion.

15. The surgical device of claim 1, wherein the shaft further comprises a plurality of teeth formed in the distal end of the shaft.

16. The surgical device of claim 15, wherein the teeth are continuous with the cutaway region and wherein the teeth form an edge defined by acutely angled edges of the cutaway region.
